(19) United States
(12) Patent Application Publication
(10) Pub. No.: US 2016/0008491 A1
(43) Pub. Date: Jan. 14, 2016

(54) METHOD FOR IMMUNE CELL TRACKING

(71) Applicant: Industrial Technology Research Institute, Chutung (TW)

(72) Inventors: Chih-Lung Chen, Taichung City (TW); Wen-Yuan Hsieh, Zhubei City (TW); Chen-Hsuan Lin, Pingtung City (TW); Shian-Jy Wang, Zhudong Township (TW)

(21) Appl. No.: 14/796,539

(22) Filed: Jul. 10, 2015

Related U.S. Application Data
(60) Provisional application No. 62/024,225, filed on Jul. 14, 2014.

(51) Int. Cl. A61K 49/18

(52) U.S. Cl. CPC A61K 49/186

(57) ABSTRACT

A method of tracking immune cells to detect immune response. The method including steps of identifying a patient having a disease associated with an organ; administering biocompatible magnetic nanoparticles into the blood stream of the patient; and obtaining a magnetic resonance image of the organ. The presence of hyperintense or hypointense spots in the magnetic resonance image indicates immune response in the patient.

### CROSS-REFERENCE TO RELATED APPLICATIONS

The present application claims the benefit of U.S. Provisional Patent Application Ser. No. 62/024,225, which was filed on Jul. 14, 2014, by Chih-Lung Chen, et al. for Method for Immune Cell Tracking and is hereby incorporated by reference.

### BACKGROUND

Immune cell tracking, which relates to monitoring immune cells' migration and accumulation, can effectively detect immune response such as immune rejection, a major cause of functional failure in patients who have received organ transplantation.

Immune response is generally monitored by periodically analyzing biopsy samples (e.g., an endomyocardial biopsy sample) to detect the presence of immune cell (e.g., T-cells and macrophages) in an organ associated with a disease.

This monitoring procedure has several drawbacks. First, as an invasive procedure, it tends to bring about adverse side effects. Further, it is prone to sampling errors that can yield false negative results. Moreover, it often fails to detect early acute or chronic rejection. Finally, it is an expensive procedure.

Immune cell tracking can also be achieved by administering to patients immune cells pre-labeled with magnetic nanoparticles. This process requires a tedious step of pre-labeling immune cells ex vivo.

There is a need to develop a simple and non-invasive method that is sensitive to track immune cells to detect early signs of disease.

### SUMMARY

Disclosed herein is a simple and non-invasive method for tracking immune cells with biocompatible magnetic nanoparticles using magnetic resonance imaging (“MRI”) scans. The method provides unexpectedly high sensitivity.

The method includes the following steps: (i) identifying a patient having a disease associated with an organ (e.g., heart, kidney, or lymph node); (ii) providing an aqueous suspension, free of particles greater than 1000 nm in size and containing biocompatible magnetic nanoparticles, (iii) administering the aqueous suspension into the blood stream of the patient; and (iv) subsequently obtaining a magnetic resonance image of the organ. Immune response is detected when the image shows the presence of hyperintense or hypointense spots (e.g., T2, T2*, or diffusion weighted MRI showing hypointense spots or T1 weighted MRI showing hyperintense spots). For example, the disease is cancer (e.g., lymphoma) or rejection of a transplanted organ (e.g., heart or kidney).

In one embodiment, the method is used to detect immune rejection, in which step (i) is to identify a patient having a transplanted organ and step (iv) is to obtain a T2-weighted magnetic resonance image of the transplanted organ. Immune rejection is then detected when the image shows the presence of hypointense spots.

The method described herein uses a contrast agent containing biocompatible magnetic nanoparticles to detect immune response with MRI technology.

The biocompatible magnetic nanoparticles each contain a superparamagnetic core that is covered by one or more biocompatible polymers, each of which has a polyethylene glycol group, a silane group, and a linker that links, via a covalent bond, the polyethylene glycol group and the silane group. Typically, these biocompatible magnetic nanoparticles each have a particle size of 10-1000 nm and a transverse magnetic relaxivity rate of 50-400. In one example, they each have a particle size of 15-200 nm and a transverse magnetic relaxivity rate of 120 to 400.

Generally, the superparamagnetic core contains an iron oxide, a cobalt oxide, a nickel oxide, or a combination thereof.

In each of the biocompatible polymers, which cover the superparamagnetic cores, the polyethylene glycol group typically has 5-1000 oxyethylene units (e.g., 10-200 oxyethylene units), and the silane group typically contains a C₁₋₁₀ alkylene group (e.g., a C₃-C₁₀ alkylene group).

The details of one or more embodiments are set forth in the description below. Other features, objects, and advantages of the embodiments will be apparent from the description and the claims.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

The method of this invention is used to track immune cells using biocompatible magnetic nanoparticles, each of which contains a superparamagnetic core covered by one or more biocompatible polymers.

The biocompatible polymers are biodegradable and nontoxic to cells. Silane-containing biocompatible polymers, which can be easily functionalized as shown below, are suitable for preparation of biocompatible magnetic nanoparticles required by this method.

An exemplary biocompatible polymer has the following formula:

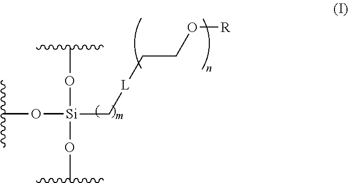

In formula (I), R is H, C₁-C₆ alkyl, C₂-C₆ alkenyl, C₂-C₆ alkynyl, C₃-C₁₀ cycloalkyl, C₁-C₁₀ heterocycloalkyl, aryl, heteroaryl, a C₁-C₁₀ carbonyl group, or a C₁-C₁₀ amine group; L is a linker; m is 1 to 10; and n is 5 to 1000.

A linker can be O, S, Si, C₁-C₆ alkylene, a carbonyl moiety containing two carbonyl groups and 2-20 carbon atoms, or a group having one of the following formula:

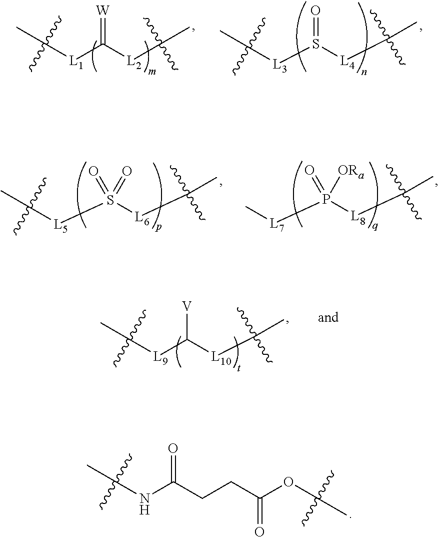

In these formula, each of m, n, p, q, and t, independently, is 1-6; W is O, S, or NR_{b}; each of L₁, L₃, L₅, L₇, and L₉, independently, is a bond, O, S, or NR_{c}; each of L₂, L₄, L₆, L₈, and L₁₀, independently, is a bond, O, S, or NR_{d}; and V is ORₑ, SR_{f}, or NR_{g}Rₕ, in which each of Rₐ, R_{b}, R_{c}, R_{d}, Rₑ, R_{f}, R_{g}, and Rₕ, independently, is H, OH, a C₁-C₁₀ oxyaliphatic radical, a C₁-C₁₀ monovalent aliphatic radical, a C₁-C₁₀ monovalent heteroaliphatic radical, a monovalent aryl radical, or a monovalent heteroaryl radical.

Another exemplary biocompatible polymer has the following formula:

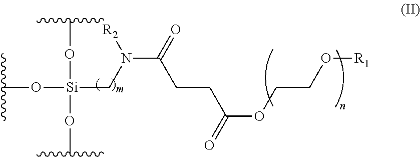

In formula (II), R₁ is H, C₁-C₆ alkyl, C₂-C₆ alkenyl, C₂-C₆ alkynyl, C₃-C₁₀ cycloalkyl, heterocycloalkyl, aryl, heteroaryl, a C₁-C₁₀ carbonyl group, or a C₁-C₁₀ amine group; R₂ is H, C₁-C₆ alkyl, C₂-C₆ alkenyl, C₂-C₆ alkynyl, C₃-C₁₀ cycloalkyl, C₁-C₁₀ heterocycloalkyl, aryl, or heteroaryl; m is 1 to 10 (e.g., 3-10); and n is 5 to 1000 (10-200). In a preferred embodiment, R₂ is H and the linker in formula (II) is

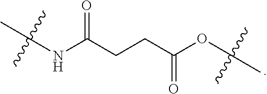 [SMILES: *NC(=O)CCC(=O)O*]

The term “aliphatic” herein refers to a saturated or unsaturated, linear or branched, acyclic, cyclic, or polycyclic hydrocarbon moiety. Examples include, but are not limited to, alkyl, alkylene, alkenyl, alkenylene, alkynyl, alkynylene, cycloalkyl, cycloalkylene, cycloalkenyl, cycloalkenylene, cycloalkynyl, and cycloalkynylene moieties. The term “alkyl” or “alkylene” refers to a saturated, linear or branched hydrocarbon moiety, such as methyl, methylene, ethyl, ethylene, propyl, propylene, butyl, butylenes, pentyl, pentylene, hexyl, hexylene, heptyl, heptylene, octyl, octylene, nonyl, nonylene, decyl, decylene, undecyl, undecylene, dodecyl, dodecylene, tridecyl, tridecylene, tetradecyl, tetradecylene, pentadecyl, pentadecylene, hexadecyl, hexadecylene, heptadecyl, heptadecylene, octadecyl, octadecylene, nonadecyl, nonadecylene, icosyl, icosylene, triacontyl, and triacotylene. The term “alkenyl” refers to a linear or branched hydrocarbon moiety that contains at least one double bond, such as —CH═CH—CH₃ and —CH═CH—CH₂—. The term “alkynyl” refers to a linear or branched hydrocarbon moiety that contains at least one triple bond, such as —C≡C—CH₃ and —C≡C—CH₂—. The term “cycloalkyl” refers to a saturated, cyclic hydrocarbon moiety, such as cyclohexyl and cyclohexylene.

The term “heteroaliphatic” herein refers to an aliphatic moiety containing at least one heteroatom (e.g., N, O, P, B, S, Si, Sb, Al, Sn, As, Se, and Ge). The term “heterocycloalkyl” refers to a cycloalkyl moiety containing at least one heteroatom. The term “oxyaliphatic” herein refers to an —O-aliphatic. Examples of oxyaliphatic include methoxy, ethoxy, n-propoxy, isopropoxy, n-butoxy, iso-butoxy, sec-butoxy, and tert-butoxy.

The term “aryl” herein refers to a C₆ monocyclic, C₁₀ bicyclic, C₁₄ tricyclic, C₂₀ tetracyclic, or C₂₄ pentacyclic aromatic ring system. Examples of aryl groups include, but are not limited to, phenyl, phenylene, naphthyl, naphthylene, anthracenyl, anthrcenylene, pyrenyl, and pyrenylene. The term “heteroaryl” herein refers to an aromatic 5-8 membered monocyclic, 8-12 membered bicyclic, 11-14 membered tricyclic, and 15-20 membered tetracyclic ring system having one or more heteroatoms (such as O, N, S, or Se). Examples of a heteroaryl group include, but are not limited to, furyl, furylene, fluorenyl, fluorenylene, pyrrolyl, pyrrolylene, thienyl, thienylene, oxazolyl, oxazolylene, imidazolyl, imidazolylene, benzimidazolyl, benzimidazolylene, thiazolyl, thiazolylene, pyridyl, pyridylene, pyrimidinyl, pyrimidinylene, quinazolinyl, quinazolinylene, quinolinyl, quinolinylene, isoquinolyl, isoquinolylene, indolyl, and indolylene.

Unless specified otherwise, aliphatic, heteroaliphatic, oxyaliphatic, alkyl, alkylene, alkenyl, alkynyl, cycloalkyl, heterocycloalkyl, aryl, and heteroaryl mentioned herein include both substituted and unsubstituted moieties. Possible substituents on cycloalkyl, heterocycloalkyl, aryl, and heteroaryl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₃-C₂₀ heterocycloalkyl, C₃-C₂₀ heterocycloalkenyl, C₁-C₁₀ alkoxy, aryl, aryloxy, heteroaryl, heteroaryloxy, amino, C₁-C₁₀ alkylamino, C₂-C₂₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amido, amidino, guanidine, ureido, thioureido, cyano, nitro, nitroso, azido, acyl, thioacyl, acyloxy, carboxyl, and carboxylic ester. On the other hand, possible substituents on aliphatic, heteroaliphatic, oxyaliphatic, alkyl, alkylene, alkenyl, and alkynyl include all of the above-recited substituents except C₁-C₁₀ alkyl. Cycloalkyl, heterocycloalkyl, aryl, and heteroaryl can also be fused with each other.

The biocompatible polymers described above include the polymers themselves, as well as their salts and solvates, if applicable. A salt, for example, can be formed between an anion and a positively charged group (e.g., amino) on a polymer. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on a polymer. Suitable cations include sodium ion, potassium ion, magnesium ion, calcium ion, and an ammonium cation such as tetramethylammonium ion. The polymers also include those salts containing quaternary nitrogen atoms. A solvate refers to a complex formed between a polymer and a pharmaceutically acceptable solvent. Examples of a pharmaceutically acceptable solvent include water, ethanol, isopropanol, ethyl acetate, acetic acid, and ethanolamine.

Scheme (I) below shows a process of preparing an exemplary silane-containing biocompatible polymer.

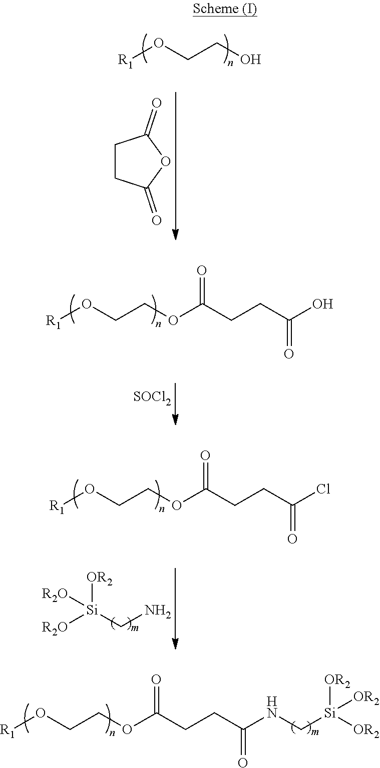

As shown in Scheme (I), alkoxyl-polyethylene glycol (molecular weight 2000) reacts with succinic anhydride in the presence of a base (e.g., dimethylaminopyridine) to form mPEG-COOH, which is subsequently converted to mPEG-COCl using thionyl chloride. Mixing mPEG-COCl with (3-aminopropyl)-triethoxysilane yields mPEG-silane.

A skilled person in the art can modify the process shown in Scheme (I) above to prepare biocompatible polymers using well-known methods. See R. Larock, Comprehensive Organic Transformations (VCH Publishers 1989); T. W. Greene and P. G. M. Wuts, Protective Groups in Organic Synthesis (3^{rd} Ed., John Wiley and Sons 1999); L. Fieser and M. Fieser, Fieser and Fieser's Reagents for Organic Synthesis (John Wiley and Sons 1994); and L. Paquette, ed., Encyclopedia of Reagents for Organic Synthesis (John Wiley and Sons 1995) and subsequent editions thereof. Specific routes that can be used to synthesize the biocompatible polymers can be found in: (a) Rist et al., Molecules, 2005, 10, 1169-1178, (b) Koheler et al., JACKS′, 2004, 126, 7206-7211; and (c) Zhang et al., Biom. Mircod., 2004, 6:1 33-40.

The biocompatible polymers described above each can be coated onto a superparamagnetic core (e.g. iron-oxide nanoparticles) via covalent bonding to form a biocompatible magnetic nanoparticle for use in a contrast agent. The superparamagnetic core has a particle size of 8 to 25 nm (e.g., 12 to 25 nm and 15 to 20 nm) and an r2 relaxivity of 120 to 250 (mM·S)⁻¹ (e.g., 150 to 230 (mM·S)⁻¹ and 170 to 210 (mM·S)⁻¹). Preparation of a superparamagnetic core is well known in the art. See Laurent et al., Chem. Rev., 2008, 108, 2064-2110.

Described below is a typical procedure to prepare superparamagnetic nanoparticles. First, iron oxide nanoparticles are suspended in toluene, followed by stirring it with mPEG-silane at room temperature for 24 hours. The resultant biocompatible magnetic nanoparticles are hydrophilic and can be extracted to a water phase and subsequently purified by ultrafiltration. The biocompatible magnetic nanoparticles thus prepared each have an r2 relaxivity of 120 to 250 (mM·S)⁻¹ (e.g., 150 to 230 (mM·S)⁻¹ and 170 to 210 (mM·S)⁻¹).

The above-described biocompatible magnetic nanoparticle can be formulated into a contrast agent, which can be administered orally. Examples of a contrast agent include emulsions, aqueous suspensions, dispersions, and solutions. If desired, certain sweetening, flavoring, or coloring agents can be added.

The biocompatible magnetic nanoparticles can be administered into patients to label immune cells (in vivo), as described in examples below. Unlike administration of immune cells pre-labeled with nanoparticles (in vivo), administration of biocompatible magnetic nanoparticles in the absence of immune cells clearly has the advantages of fewer operative steps and fewer regulatory hurdles.

Not to be bounded by any theory, the biocompatible magnetic nanoparticles, once administered to a transplant patient, is taken up by immune cells (e.g., macrophages), which are accumulated at the organ when immune response occurs. In other words, the immune cells thus labeled can be readily monitored by T1, T2, T2*, or diffusion weighted MRI, shown as hyperintense spots in a T1 weighted MRI image or shown as hypointense spots in a T2, T2*, or diffusion weighted MRI image. A procedure of conducting T1, T2*, or diffusion weighted MRI is similar to that of conducting T2 weighted MRI reported in Mol. Imaging Biol., 2011, 13(5), 825-839.

The biocompatible magnetic nanoparticles described above, when administered to patients, exhibit unexpectedly high sensitivity to MRI for tracking immune cells to monitor immune response.

The specific examples below are to be construed as merely illustrative, and not limitative of the remainder of the disclosure in any way whatsoever. Without further elaboration, it is believed that one skilled in the art can, based on the description herein, utilize the present embodiments to their fullest extent. All publications cited herein are incorporated by reference in their entirety.

### Preparation of Biocompatible Iron Oxide Nanoparticles

Two biocompatible iron oxide nanoparticles of these embodiments were prepared following the procedure described below.

### Preparation of an Iron Oxide Core

A mixture of FeCl₂.4H₂O (11.6 g; 0.058 mole), FeCl₃.6H₂O (11.6 g; 0.096 mole), and water (400 mL) was stirred at 300 rpm in a three-necked flask at 25° C. A sodium hydroxide solution (2.5 N; 170 mL) was added to the flask at a rate of 47 μl/sec, resulting a pH value of 11-12. Subsequently, oleic acid (20 mL) was added and stirred for 30 minutes, followed by addition of a 6 N HCl solution to adjust the pH value to about 1. The iron oxide core thus precipitated out of the mixture was collected by filtration, and washed with water for 4-5 times to remove excess oleic acid. It was then dried under vacuum to be used for coupling, as described below, with a biocompatible polymer.

Preparation of Biocompatible Polymer mPEG-Silane-750 and mPEG-Silane-2000

The biocompatible polymer mPEG-silane-750 was prepared following the procedure described below.

A mixture of 300 g (0.4 moles) of methoxy-PEG (mPEG, molecular weight 750), succinic anhydride (48 g; 0.48 moles) and 4-dimethylamino-pyridine (DMAP; 19.5 g; 0.159 moles) were allowed to sit in a 1000-mL round bottom flask under vacuum (20 Torts) for 2 hours. 600 mL of toluene was added to the mixture, which was then stirred at 30° C. for one day to form mPEG-COOH.

Subsequently, 36 mL (0.48 moles) of thionyl chloride was added at a rate of 1 mL/min and the mixture was stirred for 2-3 hours. Thereafter, 333.8 mL (2.4 moles) of triethylamine was added at a rate of 1 mL/min to obtain pH around 6-7. After cooling to room temperature, the mixture containing mPEG-COCl was reacted with 94.5 mL (0.4 moles) of 3-aminopropyl triethoxysilane at room temperature for at least 8 hours to yield mPEG-silane-750.

mPEG-silane-750 was precipitated after 9 L of isopropyl ether was added to the reaction mixture. The solid product was collected by filtration, re-dissolved in 500 mL of toluene, and centrifuged at 5000 rpm for 5 minutes to collect a supernatant, to which was added 9 L of isopropyl ether. Brown oily liquid was separated from the isopropyl ether and dried under vacuum to obtain the biocompatible polymer mPEG-silane-750.

The biocompatible polymer mPEG-silane-2000 was prepared following the same procedure described above using a mixture of 800 g (0.4 moles) of methoxy-PEG (mPEG, molecular weight 2000), succinic anhydride (48 g; 0.48 moles) and 4-dimethylamino-pyridine (DMAP; 19.5 g; 0.159 moles).

Coupling Each of mPEG-Silane-750 and mPEG-Silane-2000 with Iron Oxide Core

Each of biocompatible polymer mPEG-silane-750 and mPEG-silane-2000 (250 g) thus obtained was suspended in 1-1.2 L of a toluene solution containing 10 g of the iron oxide core prepared as described above. The suspension was stirred for 24 hours, followed by addition of water (1.5 L) for extraction. The extracted aqueous solution was filtered with an ultrafiltration device, washed with water, and then concentrated to 100 mL to obtain a biocompatible iron oxide nanoparticle suspension. The iron oxide nanoparticle, regardless of whether it was prepared from mPEG-silane-750 or mPEG-silane-2000, is designated as iTrast.

Characterization of Biocompatible Iron Oxide Nanoparticle (iTrast)

Transmission electron microscopy (TEM) images of the biocompatible magnetic nanoparticle iTrast thus obtained were taken using a JEOL JEM-2100F FieldEmission Transmission Electron Microscopy. The images showed that iTrast had an iron oxide core of the dimension 10-12 nm.

The transverse relaxivity (r2) and longitudinal (r1) relaxivity were determined following the procedures described in US Application Publication 2012/0329129 and Mol Imaging Biol, Chen et al., 2011, 13, 825-839. iTrast was determined to have an r2 of 205.3±2.3 (mM·s)⁻¹ and an r1 of 18.6±0.5 (mM·s)⁻¹.

### Detecting Migration and Accumulation of Macrophages in Transplantation

Studies to track macrophages in transplanted organs were performed following the procedures described below.

### Heart Transplantation in Rats

The operative procedure for using the heterotopic working-heart model is described in PNAS, 2006, 103(6):1852-1857. Inbred Brown Norway (BN; RT1ⁿ) and Dark Agouti (DA; RT1^{a}) rats were obtained from Harlan Laboratories Inc. (Indianapolis, Ind.). Allogeneic transplantation between different strains of rats (DA→BN) resulted in rejection, whereas syngeneic transplantation between the same strains of rats (DA→*DA or BN→BN) caused no rejection. The rejection grade of the heart grafts was determined histopathologically according to the guidelines described in J. Heart Lung Transplant., 1998, 17, 754-760 and J. Heart Transplant., 1990, 9, 587-593.

One day after the heart transplantation, each rat was intravenously injected with 3 mg/kg iTrast nanoparticles. It was observed that macrophages were heterogeneously distributed in the acutely rejected rat heart. Unexpectedly, in vivo MRI, conducted at day 6 post operation, indicated that macrophages labeled with iTrast nanoparticles accumulated at the allograft heart.

Histopathology confirmed an epicardium-to-endocardium progression pattern. More specifically, as rejection progressed over time, macrophage infiltration spreaded toward the inner part of the myocardium.

H&E and Perl's iron staining was performed on tissues from heart grafts harvested after in vivo MRI. Histological and immunohistochemical analyses of the grafts showed that iron-containing cells depicted by Perl's iron staining correlated with macrophage lineage ED1⁺ cells. The iron-containing cells correlated with ED1⁺ macrophages in the areas with more aggressive immune cell infiltration and disrupted myocardial integrity as revealed by H&E staining.

### Kidney Transplantation in Pigs

Major Histocompatibility Complex (MHC)-mismatched pigs were treated with high-dose tacrolimus for 12 days. Kidney allografts (n=5) were then transplanted into these pigs. As expected, at day 14, all isolated kidney allografts were rejected as the serum creatinine concentration doubled compared to that at day 0.

One day after the kidney transplantation, each pig was intravenously injected with 3 mg/kg or 6 mg/kg iTrast particles. Accumulated macrophages labeled by nano-sized iTrast in the rejected kidney were unexpectedly detected by in vivo MRI at days 3, 6, 9, 12, and 16.

Indeed, it was also found that iTrast at both 3 mg/kg and 6 mg/kg enhanced the hypointense spots around cortex at day 9 and day 6, respectively, compared with serum creatinine, indicating immune rejection of all isolated kidneys.

### Tracking Macrophages on Lymph Node

iTrast was studied to detect morphology change of a lymph node according to procedure shown below.

A mouse melanoma metastasis model using B16-F10 cells was induced in the forepaw. iTrast (2, 4, and 6 mg Fe/Kg) was administered to the mice during the tumor development, and the animals were repeatedly evaluated by MRI T2, T2*, and diffusion weighted imaging (i.e., T2WI, T2*WI, DWI) after the iTrast administration.

It was found that the tumor stages significantly affected the labeling results. When iTrast was given intravenously at a dosage of 4 mg Fe/kg, a transient labeling was unexpectedly observed in the sentinel and subsequent lymph nodes of animals having an early-stage tumor. The degree of transient signals was weakened in these lymph nodes when the tumor reached a late stage. Histology confirmed that the early-stage tumor was non-metastatic while the late-stage tumor was metastatic.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the described embodiments, and without departing from the spirit and scope thereof, can make various changes and modifications of the embodiments to adapt it to various usages and conditions. Thus, other embodiments are also within the claims. It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A method of tracking immune cells, the method comprising:
identifying a patient having a disease associated with an organ;
providing an aqueous suspension containing biocompatible magnetic nanoparticles, the aqueous suspension being free of particles having a size greater than 1000 nm, the biocompatible magnetic nanoparticles each containing a superparamagnetic core that is covered by one or more biocompatible polymers, each of which has a polyethylene glycol group, a silane group, and a linker linking, via a covalent bond, the polyethylene glycol group and the silane group;
administering the aqueous suspension into the blood stream of the patient; and
after the administration step, obtaining a magnetic resonance image of the organ, wherein the presence of hyperintense or hypointense spots in the magnetic resonance image indicates immune response in the patient.

2. The method of claim 1, wherein the magnetic resonance image is a T2 or T2* weighted magnetic resonance image.

3. The method of claim 1, wherein the disease is cancer or rejection of a transplanted organ.

4. The method of claim 3, wherein the transplanted organ is heart or kidney.

5. The method of claim 3, wherein the cancer is lymphoma.

6. The method of claim 1, wherein the organ is heart, kidney, or lymph node.

7. The method of claim 1, wherein the superparamagnetic core contains an iron oxide, a cobalt oxide, a nickel oxide, or a combination thereof; the polyethylene glycol group has 5-1000 oxyethylene units; the silane group contains a C₁₋₁₀ alkylene group; and the linker is O, S, Si, C₁-C₆ alkylene, a carbonyl moiety containing two carbonyl groups and 2-20 carbon atoms, or a group having one of the following formula: 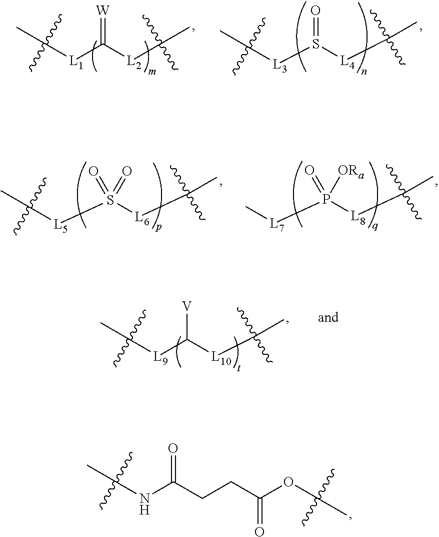
in which each of m, n, p, q, and t, independently, is 1-6; W is O, S, or NR_{b}; each of L₁, L₃, L₅, L₇, and L₉, independently, is a bond, O, S, or Na_{b}; each of L₂, L₄, L₆, L₈, and L₁₀, independently, is a bond, O, S, or NR_{d}; and V is ORₑ, SR_{f}, or NR_{g}Rₕ, each of Rₐ, R_{b}, R_{c}, R_{d}, Rₑ, R_{f}, R_{g}, and Rₕ, independently, being H, OH, a C₁-C₁₀ oxyaliphatic radical, a C₁-C₁₀ monovalent aliphatic radical, a C₁-C₁₀ monovalent heteroaliphatic radical, a monovalent aryl radical, or a monovalent heteroaryl radical.

8. The method of claim 7, wherein the biocompatible magnetic nanoparticles each have a particle size of 10-1000 nm and a transverse magnetic relaxivity rate of 50 to 400.

9. The method of claim 8, wherein the biocompatible magnetic nanoparticles each have a particle size of 15-200 nm and a transverse magnetic relaxivity rate of 120 to 400.

10. The method of claim 1, wherein the superparamagnetic core is a superparamagnetic iron oxide nanoparticle; the polyethylene glycol group has 10 to 200 oxyethylene units; the silane group contains C₃-C₁₀ alkylene; and the linker is a carbonyl moiety of the following formula:

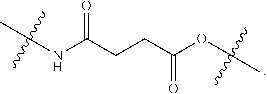 [SMILES: *NC(=O)CCC(=O)O*]

11. The method of claim 10, wherein the biocompatible magnetic nanoparticles each have a particle size of 1-1000 nm and a transverse magnetic relaxivity rate of 50 to 400.

12. The method of claim 11, wherein the biocompatible magnetic nanoparticles each have a particle size of 15-200 nm and a transverse magnetic relaxivity rate of 120 to 400.

13. The method of claim 1, wherein the biocompatible magnetic nanoparticles each have a particle size of 1-1000 nm and a transverse magnetic relaxivity rate of 50 to 400.

14. The method of claim 13, wherein the biocompatible magnetic nanoparticles each have a particle size of 15-200 nm and a transverse magnetic relaxivity rate of 120 to 400.

15. The method of claim 1, wherein the superparamagnetic core is covered by one or more biocompatible polymers having the following formula: 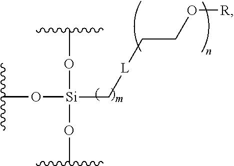
in which
R is H, C₁-C₆ alkyl, C₂-C₆ alkenyl, C₂-C₆ alkynyl, C₃-C₁₀ cycloalkyl, C₁-C₁₀ heterocycloalkyl, aryl, heteroaryl, a C₁-C₁₀ carbonyl group, or a C₁-C₁₀ amine group;
L is a linker;
m is 1 to 10; and
n is 5 to 1000.

16. The method of claim 15, wherein the linker is O, S, Si, C₁-C₆ alkylene, a carbonyl moiety containing two carbonyl groups and 2-20 carbon atoms, or a group having one of the following formula: 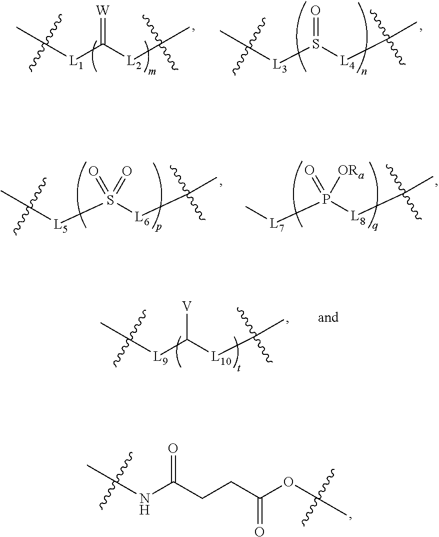
in which each of m, n, p, q, and t, independently, is 1-6; W is O, S, or NR_{b}; each of L₁, L₃, L₅, L₇, and L₉, independently, is a bond, O, S, or NR_{c}; each of L₂, L₄, L₆, L₈, and L₁₀, independently, is a bond, O, S, or NR_{d}; and V is ORₑ, SR_{f}, or NR_{g}Rₕ, each of Rₐ, R_{b}, R_{c}, R_{d}, Rₑ, R_{f}, R_{g}, and Rₕ, independently, being H, OH, a C₁-C₁₀ oxyaliphatic radical, a C₁-C₁₀ monovalent aliphatic radical, a C₁-C₁₀ monovalent heteroaliphatic radical, a monovalent aryl radical, or a monovalent heteroaryl radical.

17. The method of claim 1, wherein the superparamagnetic core is covered by one or more biocompatible polymers having the following formula: 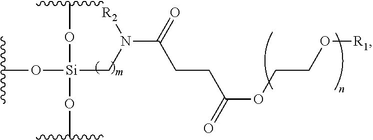
in which
R₁ is H, C₁-C₆ alkyl, C₂-C₆ alkenyl, C₂-C₆ alkynyl, C₃-C₁₀ cycloalkyl, C₁-C₁₀ heterocycloalkyl, aryl, heteroaryl, a C₁-C₁₀ carbonyl group, or a C₁-C₁₀ amine group;
R₂ is H, C₁-C₆ alkyl, C₂-C₆ alkenyl, C₂-C₆ alkynyl, C₃-C₁₀ cycloalkyl, C₁-C₁₀ heterocycloalkyl, aryl, or heteroaryl;
m is 1 to 10; and
n is 5 to 1000.

18. The method of claim 17, wherein the biocompatible magnetic nanoparticles each have a particle size of 10-1000 nm and a transverse magnetic relaxivity rate of 50 to 400.

19. The method of claim 18, wherein the biocompatible magnetic nanoparticles each have a particle size of 15-200 nm and a transverse magnetic relaxivity rate of 120 to 400.

20. The method of claim 19, wherein the disease is rejection of a transplanted heart or kidney; the superparamagnetic core is a superparamagnetic iron oxide nanoparticle; the polyethylene glycol group has 10 to 200 oxyethylene units; the silane group contains C₃-C₁₀ alkylene; the linker is a carbonyl moiety of the following formula: 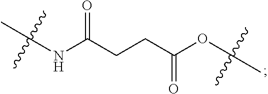 [SMILES: *NC(=O)CCC(=O)O*]
and the magnetic resonance image is a T2 or T2* weighted magnetic resonance image.

21. The method of claim 17, wherein R₁ is H; R₂ is H, C₁-C₆ alkyl, a C₁-C₁₀ carbonyl group, or a C₁-C₁₀ amine group; m is 3 to 10; and n is 10 to 200.

22. The method of claim 21, wherein the biocompatible magnetic nanoparticles each have a particle size of 10-1000 nm and a transverse magnetic relaxivity rate of 50 to 400.

23. The method of claim 22, wherein the biocompatible magnetic nanoparticles each have a particle size of 15-200 nm and a transverse magnetic relaxivity rate of 120 to 400.
